(19) United States
(12) Patent Application Publication
(10) Pub. No.: US 2022/0120921 A1
(43) Pub. Date: Apr. 21, 2022

(54) HIGH RESOLUTION X-RAY DETECTOR SYSTEM

(71) Applicant: Brown Universtiy, Providence, RI (US)

(72) Inventors: Theodore F. MORSE, Providence, RI (US); Angus Ian KINGON, Warren, RI (US); Nicholas Alexander MOSTOVYCH, Silver Spring, MD (US)

(21) Appl. No.: 17/503,761

(22) Filed: Oct. 18, 2021

Related U.S. Application Data
(60) Provisional application No. 63/092,651, filed on Oct. 16, 2020.

(51) Int. Cl. G01T 1/20; A61B 6/00

(52) U.S. Cl. CPC G01T 1/20185; G01N 23/04; A61B 6/4233

(57) ABSTRACT

An X-ray detector includes a scintillation plate and sensors, the scintillation plate having a glass capillary array with scintillation material filling, wherein the glass capillary array with scintillation material filling is mated with two high volume, low cost, CMOS sensors, and wherein the glass capillary array is arranged diagonally to mate with active parts of the two high volume, low cost, CMOS sensors.

### CROSS REFERENCE TO RELATED APPLICATIONS

This application claims benefit from U.S. Provisional Patent Application Ser. No. 63/092,651, filed Oct. 16, 2020, which is incorporated by reference in its entirety.

### STATEMENT REGARDING GOVERNMENT INTEREST

This invention was made with government support under grant number 1819978 awarded by the National Science Foundation and grant number R41 DE029386 awarded by National Institutes of Health. The government has certain rights in the invention.

### BACKGROUND OF THE INVENTION

The present invention relates generally to X-ray systems, and more particularly to a high resolution X-ray detector system.

In general, coherent bundles of scintillating fibers are useful for detecting x-rays and are placed opposed to a complementary metal-oxide-semiconductor (CMOS) sensor. Incident x-rays activate the scintillators in individual fibers, which then emit visible light to the camera, which then generates an image. Coherent bundles are used extensively in medical, scientific and engineering applications. Particularly in the medical imaging field, coherent bundles are instrumental in creating images later used to diagnose cancer, heart disease and other ailments. In engineering fields, parts may be imaged to determine if they have micro-fractures, which may lead to premature failure of the part, such as turbine blades in a jet engine. In domestic security, x-ray imaging is used for scanning of packages, luggage and persons for weapons and contraband.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features an X-ray system including an X-ray source, and an X-ray detector, the X-ray detector comprising a scintillation plate and sensors, the scintillation plate having a glass capillary array with scintillation material filling.

In another aspect, the invention features an X-ray detector including a scintillation plate and sensors, the scintillation plate having a glass capillary array with scintillation material filling, wherein the glass capillary array with scintillation material filling is mated with two high volume, low cost, CMOS sensors, and wherein the glass capillary array is arranged diagonally to mate with active parts of the two high volume, low cost, CMOS sensors.

In still another aspect, the invention features an X-ray detector including a scintillation plate having an optical taper that matches a scintillation area to active areas of photodetectors, the optical taper resulting from a fused optical fiber bundle that is tapered in cross-section.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 illustrates an exemplary system.

FIG. 2 illustrates a first embodiment of an exemplary X-ray detector.

FIG. 3 illustrates a second embodiment of an exemplary X-ray detector.

FIG. 4 illustrates a third embodiment of an exemplary X-ray detector.

FIG. 5 illustrates a fourth embodiment of an exemplary X-ray detector.

FIG. 6 illustrates a prospective view of four optical diagonal arrays interfaced with four CMOS sensors.

FIG. 7 illustrates an aerial view of four exemplary fiber optic diagonal arrays interfaced with four exemplary CMOS sensors.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

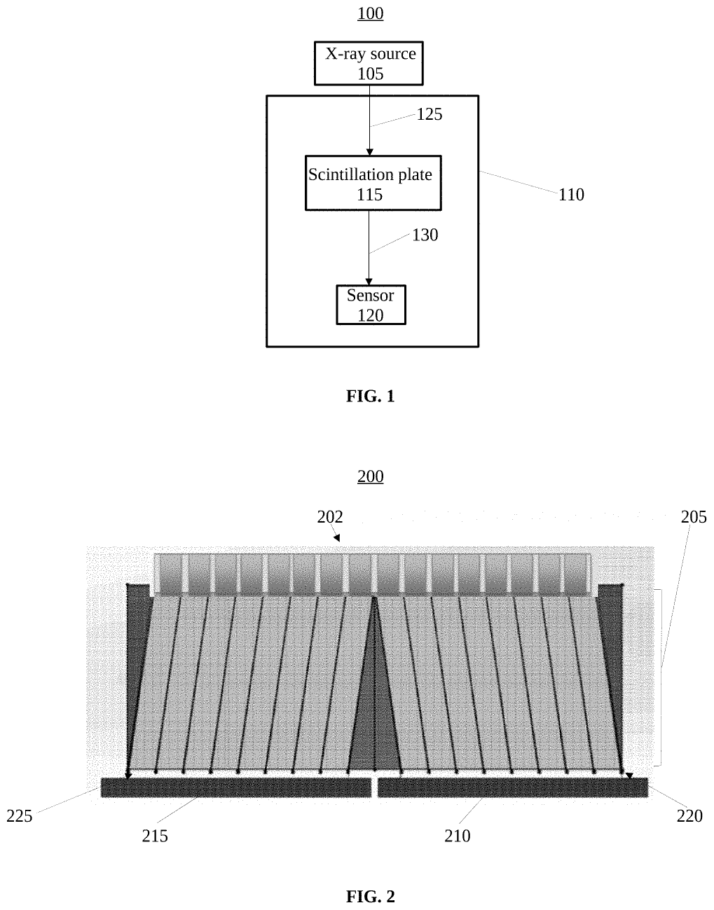

As shown in FIG. 1, an exemplary X-ray system 100 includes an X-ray source 105 and an X-ray detector 110. The X-ray detector 110 includes a scintillation plate 115 and a sensor 120. The scintillator plate 115 coverts X-ray light 125 from the X-ray source 105 to visible light 130. The visible light 130 is then converted to electrical signals by the sensor 120.

Referring to FIG. 2, a first embodiment of an exemplary X-ray detector 200 includes a scintillator plate 202 having a glass capillary array 205 with scintillation material filling, mated with two high volume, low cost, CMOS sensors 210, 215. The method to manufacture a coherent bundle of scintillating fibers is disclosed in U.S. Pat. No. 9,611,168, incorporate herein in its entirety.

Here, the scintillation plate 202 has a spatial resolution of 5 microns. To capture this 5 micron resolution at an image, the scintillator plate 202 must be mated to a photodetector sensor 210, 215 that is of equal or higher spatial resolution. To overcome this problem, the glass capillary array 205 is arranged diagonally to mate with an active part 220, 225 of the CMOS sensor 210, 215.

It should be noted that although the scintillator plate 202 in based upon a glass capillary array 205, other forms of scintillators plates may be used. For example, referring to FIG. 3, a second exemplary embodiment of an X-ray detector 300 includes a CsI:Tl scintillator plate 305 and diagonal glass fiber optic arrays 310 mated to an active section 315 of 320 CMOS photodetector 320.

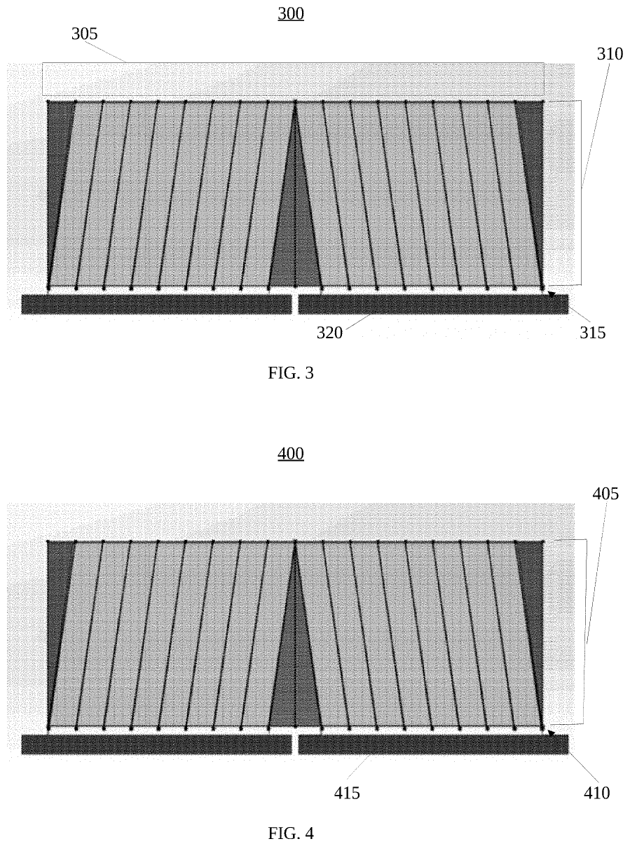

As shown in FIG. 4, a third exemplary embodiment of an X-ray detector 400 a diagonal scintillator plate 405 that includes diagonal glass capillary arrays filled with scintillator material that are mated with an active area 410 of a CMOS photodetector 415. Thus, in this embodiment, the scintillator plate and the diagonals are integrated into one structure. And instead of diagonals of fused fiber optical arrays, the diagonals are fused capillary tubes (hollow) which are filled with scintillator material. They are designed to both convert the incoming X-rays to photons, and also confine the photons to the capillary cores to preserve a desired resolution.

In FIG. 5, a fourth exemplary embodiment of an X-ray detector 500 includes a scintillation plate 505 having an optical taper, i.e., a fused optical fiber bundle that is tapered in cross-section, which matches the scintillation area to the active areas 510, 515 of the photodetectors 520, 525.

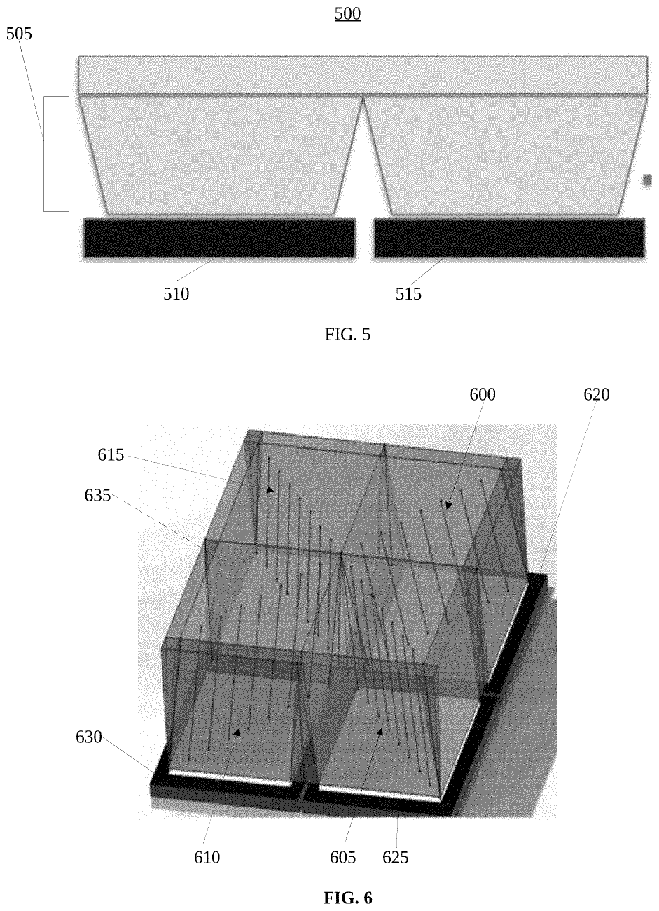

As shown in FIG. 6, In a fifth embodiment, four optical diagonal glass capillary arrays 600, 605, 610, 615 are interfaced with four CMOS sensors 360, 625, 630, 635. With such an arrangement, a full image object may be imaged by the active sensor areas of the four CMOS sensors 620, 625, 630, 635.

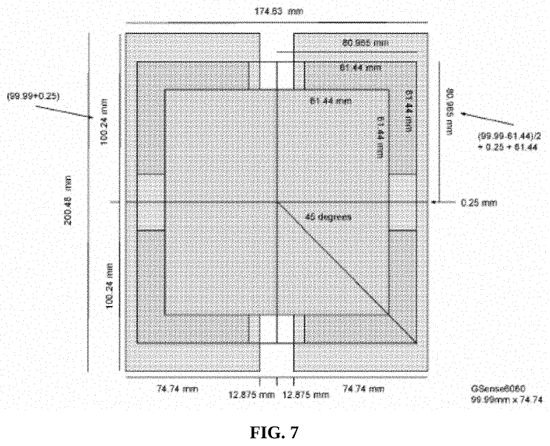

In FIG. 7, an aerial view of four exemplary fiber optic diagonal arrays interfaced with four exemplary CMOS sensors is illustrated.

In summary, in an aspect, the X-ray detector system of the present invention includes three primary elements. A first element is a geometrically confined scintillator plate, such as a glass capillary array with a high-aspect-ratio pore structure that is infiltrated with a high yield scintillating material. This first element is interfaced with an optical fiber bundle (second element), the fiber optic bundle having slightly diagonal fibers, which allows for four low cost, high resolution CMOS censors to be grouped together, to achieve a large active sensing area. These four CMOS sensors make up a third key element. The four CMOS sensors are interfaced with the diagonal fiber optic bundle in such a way that they can be grouped together without a loss of detection area that would result from edge electronics and connections associated with the non-active sensing area of each smaller commercial CMOS sensor. This enables the integration of a high resolution geometrically confined scintillator plate with a large active area of micron-scale resolving imagers, without the need for a prohibitively expensive large area CMOS detector.

For example, in one implementation, the system is a 5″×5″ (12.7 cm×12.7 cm) high resolution X-ray detector system capable of imaging large objects with resolution better than 10 microns.

The system of the present invention enables high resolution X-ray imaging of large objects. It can be used in various medical applications ranging from medical radiography and fluoroscopy. Other applications include, for example, commercial and defense high-resolution X-ray imaging modalities, and so forth.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. An X-ray system comprising:
an X-ray source; and
an X-ray detector, the X-ray detector comprising a scintillation plate and sensors, the scintillation plate having a glass capillary array with scintillation material filling.

2. The X-ray system of claim 1 wherein the glass capillary array with scintillation material filling is mated with two high volume, low cost, CMOS sensors.

3. The X-ray system of claim 2 wherein the glass capillary array is arranged diagonally to mate with active parts of the two high volume, low cost, CMOS sensors.

4. The X-ray system claim 1 wherein the X-ray detector comprises a CsI:Tl scintillator plate and diagonal glass fiber optic arrays mated to an active section of a CMOS photodetector.

5. The X-ray system claim 1 wherein the X-ray detector comprises a diagonal scintillator plate comprising diagonal glass capillary arrays filled with scintillator material that are mated with an active area of a CMOS photodetector.

6. The X-ray system claim 1 wherein the X-ray detector comprises a scintillation plate having an optical taper that matches a scintillation area to active areas of photodetectors.

7. The X-ray system of claim 6 wherein the scintillation plate comprises a fused optical fiber bundle that is tapered in cross-section.

8. An X-ray detector comprising:
a scintillation plate and sensors, the scintillation plate having a glass capillary array with scintillation material filling,
wherein the glass capillary array with scintillation material filling is mated with two high volume, low cost, CMOS sensors, and
wherein the glass capillary array is arranged diagonally to mate with active parts of the two high volume, low cost, CMOS sensors.

9. An X-ray detector comprising:
a scintillation plate having an optical taper that matches a scintillation area to active areas of photodetectors, the optical taper resulting from a fused optical fiber bundle that is tapered in cross-section.
